(19) United States
(12) Patent Application Publication
(10) Pub. No.: US 2023/0036932 A1
(43) Pub. Date: Feb. 2, 2023

(54) SENSOR FOR DETECTION OF GAS AND METHODS FOR MANUFACTURING

(71) Applicant: SENTEC AG, Therwil (CH)

(72) Inventors: Dominik RUDMANN, Basel (CH); Peter Matthias SCHUMACHER, Kirchlindach (CH); Marco ZUMBRUNN, Volketswil (CH); Manuel ENGLER, Freiburg (DE); Amirhossein DROUDIAN, Thalwil (CH); Victoria DE LANGE, Olten (CH)

(21) Appl. No.: 17/786,984

(22) PCT Filed: Dec. 20, 2019
(86) PCT No.: PCT/EP2019/086823

§ 371 Date: Jun. 17, 2022

(51) Int. Cl. G01N 21/3504; G01N 33/497; G01N 33/49

(52) U.S. Cl. CPC G01N 21/3504; G01N 33/497; G01N 33/4925

(57) ABSTRACT

The invention concerns sensors (1) for detection of gas, in particular sensors for detection of transcutaneous gas such as CO₂, and methods for manufacturing a sensor (1). The sensor (1) comprises at least one radiation source (3) for emitting radiation, at least one detector (4) for detection of radiation emitted by the radiation source (3), and at least one measurement chamber (6) for receiving the sample gas. The radiation source (3), the detector (4), and the measurement chamber (6) are arranged such that at least a part of the radiation propagates along a path passing through the measurement chamber (6). The sensor (1) further comprises a casing (7), wherein the radiation source (3), the detector (4), the measurement chamber (6) are arranged. The sensor (1) has a contact face (8) which is directable towards a measuring site and the sensor (1) has at least one gas-access channel (9) enabling gas to migrate from the contact face (8) into the measurement chamber (6). The casing (7) comprises a, preferably metallic, material having a high thermal conductivity, preferably more than 10 W/m/K.

The invention concerns sensors for detection of gas, in particular sensors for detection of CO₂, and methods for manufacturing a sensor.

Current systems for measuring gas employ a variety of measurement principles. In medical technology, predominantly electrochemical sensors are used for measurement of gases that have diffused transcutaneously, i.e. that have diffused through the skin of a human patient or of an animal. Current sensors for measurement of transcutaneous CO₂ are very sensitive and show good response times. Electrochemical sensors for measurement of transcutaneous gases are for example known from WO 2008/132205.

In certain clinical settings involving extracorporeal blood circulation, it is furthermore possible to measure blood gases directly from the blood stream of a patient by using transcutaneous gas sensors.

Optical sensors for measurement of transcutaneous gases are for example known from WO 2015/011103.

Transcutaneous gases may refer to a mixture of gases. Transcutaneous gases typically can be CO₂, O₂, H₂O, N₂, and anesthetic gases.

Instead of transcutaneous gases, such a sensor is also capable of measuring the concentration of non-transcutaneous gases, for example, gases from an extracorporeal blood stream. In the context of the present application, the term “sample gas” refers to gas that can be measured by the sensor irrespective of its origin. The expression “gas to be measured” used in this document is equivalent to the term “sample gas”.

Within this application the term contact face shall denote the face of the sensor in direct or indirect contact with the measurement site, which typically is the skin of a patient or a vessel encompassing a blood stream. One or several additional gas-permeable membranes and contact liquids or gels may be present between the sensor's contact face and the measurement site at least during a measurement. The contact face typically is formed primarily by a part of the surface of a casing and by an area allowing gas diffusion into the measurement chamber. For example, the contact face may be formed by a combination of the surfaces of a part of a casing, of gas-permeable membranes such as gas-access channel seals, and of zones related to sensor assembly such as epoxy. A casing having a contact face means a casing of which a certain surface section will form at least a part of the contact face once the sensor is fully assembled.

One of the main technological challenges in transcutaneous gas sensing is related to the fact that the rates, at which the gases permeate through tissue and skin, are very low. Thus, when a sensor for measurement of transcutaneous gases relies on equilibration of the concentrations of the gases in a measurement chamber with the respective concentrations in the skin, it must have an extremely small volume of such a measurement chamber to achieve acceptable response (equilibration completion) times. Moreover, with an optical sensor having such a small measurement chamber, guiding the optical radiation to and through the measurement chamber at sufficient optical intensity is a challenge. Another constraint is the small area of the measuring site, which is typically an earlobe of a grown-up patient or the thigh of a neonatal baby.

Since usually only a small amount of sample gas is available, the sensor is extremely sensitive with respect to possible disturbances, such as unwanted leakage of gas or faulty absorption of measurement radiation.

Within this application the term measurement radiation means radiation generated by a radiation source, where the radiation propagates through the measurement chamber and is detectable by a detector. Typically, at least a fraction of the measurement radiation propagating through the measurement chamber can be absorbed by sample gas present in the measurement chamber. When the sensor is a sensor for measuring CO₂, measurement radiation preferably has wavelengths in the range from 3.5 to 4.5 μm or within that range, but other wavelengths or wavelength bands may be employed to measure other gases or other properties of CO₂.

Sample gas being present in a measurement chamber can give rise to absorption of measurement radiation and therewith to a measurement signal. The measurement chamber is mainly defined by its confining surfaces except for the (typically small) locations, where sample gas needs to access or leave the measurement chamber. In such locations, there may be no material surface and the measurement chamber boundaries are defined as the smooth extensions of the neighboring material surfaces, corresponding to the material surfaces one would expect to have in the absence of gas-access openings.

A measurement chamber typically is defined by the surfaces of several components. For example, a measurement chamber may be formed by parts of surfaces of an optical module support, of a mirror, of a radiation entrance window and of a radiation exit window.

A sensor for detection of gas may comprise a mirror, which forms a part of the measurement chamber and which has at least a part of one face being reflective for measurement radiation. The mirror may be added to the assembly at a suitable stage, for example after application of a surface coating onto some of the surfaces of the measurement chamber. This mirror may be porous, such that it is permeable for atoms and molecules of sample gas, while it may be liquid tight. If such a porous and liquid-tight mirror forms a part of the sensor's contact face, transcutaneous gas needs to migrate only a very short distance from the skin surface to the measurement chamber, which is advantageous because it helps to keep the sensor's response time short.

An optical module support may be contained within the sensor. The optical module support may carry at least one of radiation source, detector, wavelength-sensitive element, mirror. These constituents may be arranged on or within the optical module support. The optical module support may also encompass at least a part of a measurement chamber. An optical module support equipped with such sensor constituents forms an optical module. The optical module may also comprise further components or features, for example radiation entrance and exit windows, radiation-window seals along radiation entrance and/or exit windows, coatings, adhesives, membranes, or gas-access channels. The optical module may serve as an optical measurement unit, such that—when appropriately connected and driven—it is capable of generating measurement radiation and of providing an absorption measurement signal. An optical module may form the basis of a single-parameter sensor, or it may be combined with other sensing units and/or further optical modules to form a multi-parameter sensor.

One or several gas-access channels may exist within the sensor. They create a diffusion path for molecules of the sample gas leading from the measurement chamber to the sensor surface, typically to the sensor's contact face. During a measurement, the gas-access channels ensure that sample gas can diffuse into or out of the measurement chamber efficiently, which allows gas concentration equilibration with the ambient concentrations (typically with concentrations in the skin) within a useful response time. Preferably, gas-access channels are not completely filled with solids or liquids, such that sample gas can easily migrate through them.

During measurements on patients, a high amount of humidity may be present in the measurement chamber. Since liquid water tends to absorb and deflect measurement radiation, water condensation within the measurement chamber must be avoided.

To capture water molecules from a sample gas mixture and to reduce humidity, a water trap may be integrated into the sensor or into a disposable part of the sensor. However, assembly is challenging due to the small size of the sensor and the water trap only can have a limited capacity.

It is an object of the present invention to avoid the drawbacks of the state of the art and in particular to provide a robust sensor for detection of gas, in particular for determining the concentration of a gas species in small volumes of a gas mixture, and a method for detection of gas, in particular for determining the concentration of a gas species in small volumes of a gas mixture, where no frequent calibrations are required, accurate results are delivered within acceptable response times, and manufacture is easy and inexpensive.

Response time in this context is understood as the time a sensor requires, once applied on a patient, to reach 90% of a stable measurement value. Acceptable response times are shorter than 10 minutes, preferably shorter than 5 minutes and more preferably shorter than 2.5 minutes.

According to the invention, these objects are accomplished by sensors and methods according to the independent patent claims.

According to a first aspect of the invention a sensor for detection of gas, preferably of transcutaneous gas and in particular of gaseous CO₂, is provided.

The sensor comprises at least one radiation source for emitting radiation, at least one detector for detection of radiation emitted by the source and at least one measurement chamber for receiving the sample gas, preferably arranged on or in an optical module support to form an optical module.

The radiation source, the detector, and the measurement chamber are arranged such that at least a part of the radiation propagates along a path. The path passes through the measurement chamber.

Preferably, the sensor further comprises a mirror that is arranged such that the radiation path involves a reflection at the mirror. Preferably, the mirror is arranged on or in an optical module support and in particular in the same optical module support as the radiation source and the detector.

The sensor further comprises a casing. The source, detector, measurement chamber, and optionally further components such as an optical module or a mirror are arranged in the casing. The sensor, in particular the casing, has a contact face which is directable towards a measuring site and which is intended to be in close contact with the skin of a patient during a transcutaneous measurement.

The sensor may comprise at least one gas-access channel enabling sample gas, preferably transcutaneous gas, to migrate from the contact face into the measurement chamber. Preferably, the sensor comprises more than one gas-access channel, for example two, three, or four gas-access channels. Preferably, at least a part of the gas-access channel(s) is arranged on or in an optical module support.

The casing comprises or entirely consists of a material having a high thermal conductivity. The thermal conductivity preferably exceeds 10 W/m/K. The material is preferably metallic or ceramic. Metal alloys such as aluminum, copper, or titanium alloys, for example brass, or ceramics based on alumina, zirconia, silicon or boron nitride or silicon carbide are particularly well suited.

The casing may be used for efficient transport of thermal energy during use. Thermal energy typically is generated on purpose by controllable heating elements, such as heating resistors, but also waste heat generated by other components such as amplifiers, microprocessors, etc. contribute to the generated thermal energy. The thermal energy produced by the heating elements and by further components needs to be transported to the contact face of the sensor for warming the skin of the patient at the measuring site. Typically, such heating elements and other heat-generating components are arranged within the sensor at locations distant from the contact face, for reasons such as proximity to electrical power supply or availability of space. A thermally well-conducting casing causes the generated heat to flow to the patient's skin through the casing rather than through the sensor's interior parts. This reduces temperature gradients within the sensor.

According to a second aspect of the invention a sensor for detection of gas, preferably of transcutaneous gas and in particular of gaseous CO₂, is provided. Preferably, the sensor is a sensor as described above.

The sensor comprises at least one radiation source for emitting radiation, at least one detector for detection of radiation emitted by the source, and at least one measurement chamber for receiving the sample gas, preferably arranged on or in an optical module support to form an optical module.

The radiation source, the detector, and the measurement chamber are arranged such that at least a part of the radiation propagates along a path. The path passes through the measurement chamber.

Preferably, the sensor further comprises a mirror for reflecting radiation emitted by the radiation source that is arranged such that the radiation path involves a reflection at the mirror. Preferably, the mirror is arranged on or in an optical module support and in particular in the same optical module support as the radiation source and the detector.

The sensor further comprises a casing. The source, the detector, the measurement chamber, and optionally further components such as an optical module or a mirror are arranged within the casing. The sensor, in particular the casing, has a contact face which is directable towards a measuring site and which is intended to be in close contact with the skin of a patient during a transcutaneous measurement.

The sensor may comprise at least one gas-access channel enabling sample gas, preferably transcutaneous gas, to migrate from the contact face into the measurement chamber. Preferably, the sensor comprises more than one gas-access channel, for example two, three, four, or more gas-access channels. As long as diameter and volume of the gas-access channels allow, many gas-access channels, such as hundreds or thousands, may be realized.

The radiation source is placed within the sensor in a source compartment, for example within a source compartment formed in an optical module support. The source compartment preferably is separated from the measurement chamber by a radiation entrance window. The source compartment typically is gas filled. A radiation-window seal along the radiation entrance window may reduce or avoid gas exchange between measurement chamber and source compartment.

The sensor comprises at least one venting channel leading from the source compartment to the environment. Preferably, at least a part of the venting channel leads through the casing. Preferably, at least a part of the venting channel leads through the optical module support, if present.

The venting channel reduces the risk of accumulation of relevant amounts of unwanted gas in the source compartment. For example, a leaky radiation-window seal along the radiation entrance window might allow sample gas to diffuse from the measurement chamber into the source compartment with time. Furthermore, unwanted gas can be generated within the source compartment, for example by decomposition of adhesives owing to irradiation from the source or by outgassing or out-diffusion of volatile atoms or molecules from sensor constituents in or near the source compartment.

The presence of a venting channel ensures a certain gas exchange with the environment, allowing unwanted gas to escape from the source compartment and therefore ensuring unbiased measurements. The rate at which unwanted gas appears in the source compartment should be lower than the rate at which unwanted gas disappears into the environment. Since the former rate typically is low, the latter rate does not need to be particularly high.

The venting channel may be formed by a tube, preferably a tube made from a metal such as steel or from a polymer. The tube may lead from the source compartment through the sensor's casing wall or alternatively to an opening in the sensor's casing or cap.

In a preferred embodiment of this aspect, the sensor comprises a venting-channel seal for sealing the venting channel. The venting-channel seal is gas permeable to some extent and thus allows unwanted gas to escape from the source compartment over time. The venting-channel seal prevents contamination like dust or particles from entering the venting channel and the source compartment, which could lead to radiation source problems or faulty measurements. Preferably, the venting-channel seal is liquid tight such that liquids and cleaning agents like application gel, sweat, or alcohols are also prevented from entering the venting channel and the source compartment under normal clinical use conditions.

Preferably the venting-channel seal allows gas in the source compartment, for example CO₂ and H₂O, to equilibrate with environmental concentrations within less than 1 month, preferably within several hours to few days.

Preferably the venting-channel seal is designed such that the rate at which unwanted gas diffuses out of the source compartment into the environment is substantially higher (e.g. by a factor of 10) than the rate at which unwanted gas appears in the source compartment, where unwanted gas appears for example due to diffusion from the measurement chamber through a radiation-window seal or due to outgassing or generation within the source compartment.

The venting-channel seal may have any suitable shape. Preferably, it is used in the form of a plug, a disk, or a cap.

Preferably, the venting-channel seal comprises or is made from a material which is applicable as a viscous liquid or a paste and which cures to a solid, gas-permeable material. Preferably, a polymer, in particular a silicone, an epoxy, acrylic, cyanoacrylate, or urethane compound, typically available as an adhesive, is used.

Additionally or alternatively, the venting-channel seal may comprise or may be made from a solid, non-porous, gas-permeable material. In particular, the solid material comprises a silicone, fluoropolymer, or polyolefin compound, for example PDMS or PTFE or PP.

Additionally or alternatively, the venting-channel seal may also comprise a porous material, such as a sintered ceramic, sintered polymer, or sintered metal, in particular a sintered fluoropolymer such as PTFE, a sintered steel, or a sintered titanium.

Additionally or alternatively, the venting-channel seal may also comprise or may be made from a combination of a supporting material having pores, voids or holes and a gas-permeable layer, such as a foil, a membrane, or a coating.

In a further preferred embodiment, the detector is placed in a detector compartment and the sensor may comprise a venting channel from the detector compartment to the environment in analogy to the venting channel for the source compartment described above. In particular, the detector venting channel may be covered by a venting-channel seal. The sensor may comprise a detector venting channel instead of or in addition to a source venting channel.

This is particularly useful if a radiation-window seal exists along the radiation exit window, which only retards gas exchange between measurement chamber and detector compartment to some extent.

According to a third aspect of the invention a sensor for detection of gas, preferably of transcutaneous gas and in particular of gaseous CO₂, is provided. Preferably, the sensor is a sensor as described above.

The sensor comprises at least one radiation source for emitting radiation, at least one detector for detection of radiation emitted by the source, a mirror for reflecting the radiation, and at least one measurement chamber for receiving the sample gas, preferably arranged on or in an optical module support to form an optical module.

The radiation source, the detector, the mirror and the measurement chamber are arranged such that at least a part of the radiation propagates along a path. The path passes through the measurement chamber and involves a reflection at the mirror.

The sensor has a contact face which is directable towards a measuring site and which is intended to be in close contact with the skin of a patient during a transcutaneous measurement.

The sensor may comprise at least one gas-access channel enabling sample gas, preferably transcutaneous gas, to migrate from the contact face into the measurement chamber. Preferably, the sensor comprises more than one gas-access channel, for example two, three, or four gas-access channels.

The mirror is arranged at a distance from the contact face.

If the mirror is not arranged at the contact face, the mirror is not strongly cooled by the skin during use. Hence, even when contacting humid sample gas the risk of water condensation on the mirror's surfaces is reduced.

For example, the mirror may be arranged on a layer of epoxy or of some other material, such that the contact face is separated from the mirror mostly or completely by that layer.

The sensor preferably comprises a casing. Sensor components, such as an optical module, are arranged in the casing. At least a part of the contact face may be formed by a part of the casing's surface. The mirror may be arranged on a part of the casing, such that the mirror and the contact face are separated at least by a part of the casing.

A casing with high thermal conductivity as described above may act as a thermal shield between the skin and the measurement chamber and/or the mirror, especially when the sensor interiors are at a warmer temperature than the contact face. Therewith, during a measurement on a patient, the skin mainly cools the casing instead of directly the mirror.

When measuring the concentration of humid sample gases, the risk for water condensation in the measurement chamber and particularly on the mirror is reduced, since the mirror is at about the same temperature as the remainder of the measurement chamber surfaces and hence is not significantly influenced by the temperature of the contact face.

Alternatively or additionally, the sensor further comprises a casing. The mirror is then arranged at a distance from the casing. The mirror is thermally decoupled from the casing. In particular, a thermally insulating layer may be arranged between the mirror and the casing.

The insulating layer preferably consists of a material having low thermal conductivity, preferably less than 10 W/m/K, more preferably less than 0.1 W/m/K. Alternatively, the insulating layer may be formed by a gas such as air.

The insulating layer further preferably has a low solubility for sample gas, such that sample gas will be absorbed by the insulating layer at most at an insignificant rate.

Preferred materials for the insulating layer include polymers such as epoxy or other potting materials.

According to a fourth aspect of the invention a sensor for detection of gas, preferably of transcutaneous gas and in particular of gaseous CO₂, is provided. Preferably, the sensor is a sensor as described above.

The sensor comprises at least one radiation source for emitting radiation, at least one detector for detection of radiation emitted by the source, a mirror for reflecting the radiation, and at least one measurement chamber for receiving the sample gas, preferably arranged on or in an optical module support to form an optical module.

The radiation source, the detector, the mirror and the measurement chamber are arranged such that at least a part of the radiation propagates along a path. The path passes through the measurement chamber and involves a reflection at the mirror.

The sensor has a contact face which is directable towards a measuring site and which is intended to be in close contact with the skin of a patient during a transcutaneous measurement.

The sensor comprises at least one gas-access channel enabling sample gas, preferably transcutaneous gas, to migrate from the contact face into the measurement chamber. Preferably, the sensor comprises more than one gas-access channel, for example two, three, or four gas-access channels.

The walls of the at least one gas-access channel are arranged distant from the mirror's edge and such that they do not lead through the mirror. Preferably, all gas-access channel walls are arranged distant from the mirror's edge and such that they do not lead through the mirror.

When the gas-access channels lead neither through nor directly around the mirror, reliable attachment and sealing of a mirror during sensor assembly is easier. For example, when the mirror is attached using an adhesive, there is a reduced risk of adhesives flowing into gas-access channels and/or into the measurement chamber. Adhesives flowing into gas-access channels or into the measurement chamber may hinder or even block exchange of sample gas or may excessively absorb measurement radiation.

Furthermore, when a thermally insulating layer is arranged between mirror and casing, preferably as described above, it may be beneficial to apply this thermally insulating layer in the form of a liquid adhesive that will transform into a solid material upon curing, for example in the form of an epoxy adhesive.

The openings of the gas-access channels in the contact face preferably are arranged on a closed line surrounding the mirror, in particular on a circle. This simplifies sensor assembly (in particular mirror attachment) and allows collecting gas from an extended gas-collection area.

In a preferred embodiment of this aspect, a portion of the gas-access channel may lead along a part of the mirror, in particular along a part of the inner face of the mirror, i.e. the face directed to the measurement chamber.

The creation of gas-access channels and the attachment of the mirror may be separate and independent process steps, which reduces the complexity and demands on manufacturing and increases the reliability of the manufactured parts.

Preferably, the gas-access channel originates in the measurement chamber, then runs along the mirror's inner face but not as far as to the mirror's edge. This enables sealing the mirror in a liquid-tight way, as described above. The part of the gas-access channel leading along the inner face of the mirror is well accessible during manufacturing and before the mirror is attached, which enables the creation of small openings of the gas-access channels into the measurement chamber. The provision of large openings would lead to excessive loss of measurement radiation into the channels and hence poor measurement signal quality.

Furthermore, those sections of the gas-access channels not leading along the inner face of the mirror preferably lead through material having a high thermal conductivity, e.g. through the bulk material of the optical module support. The risk for cold spots on the gas-access channel surfaces giving rise to water condensation then is reduced.

Preferably, gas-access channels, measurement chamber, and mirror are arranged on or in an optical module support.

Preferably, an optical module support having a high thermal conductivity is employed. The mirror may be attached to that optical module support, and the gas-access channels always run at the surface of or within the optical module support. Therewith, inner surfaces of the gas-access channels and of the measurement chamber are easier to keep at a homogeneous temperature.

According to a fifth aspect of the invention a sensor for detection of gas, preferably of transcutaneous gas and in particular of gaseous CO₂, is provided. Preferably, the sensor is a sensor as described above.

The sensor comprises at least one radiation source for emitting radiation, at least one detector for detection of radiation emitted by the source, a mirror for reflecting the radiation, and at least one measurement chamber for receiving the sample gas, preferably arranged on or in an optical module support to form an optical module.

The radiation source, the detector, the mirror and the measurement chamber are arranged such that at least a part of the radiation propagates along a path. The path passes through the measurement chamber and involves a reflection at the mirror.

The sensor has a contact face which is directable towards a measuring site and which is intended to be in close contact with the skin of a patient during a transcutaneous measurement.

The sensor preferably comprises at least one gas-access channel enabling sample gas, preferably transcutaneous gas, to migrate from the contact face into the measurement chamber. Preferably, the sensor comprises more than one gas-access channel, for example two, three, or four gas-access channels.

The mirror consists of a deformable material. Preferably, the mirror further is highly reflective for measurement radiation, is inert, non-porous, and/or thermally well conducting, and most preferably it possesses all of these properties.

The mirror may comprise and preferably consist of a solid but deformable material. The material may be relatively soft, having hardness preferably of less than 100 HV5, more preferably of less than 60 HV5 and most preferably of less than 30 HV5. The material then may be deformed mechanically and plastically with relatively low force, enabling mirror attachment by deformation. Preferably, the deformable mirror can be plastically deformed at temperatures smaller than 150° C., more preferably at lower temperatures and most preferably at room temperature.

The mirror preferably exhibits a high reflectivity for measurement radiation, at least on its inner face, which is the face directed towards the measurement chamber. Preferably, the reflectivity of the mirror for wavelengths in the range of the wavelengths of measurement radiation exceeds 80%, more preferably exceeds 92%, and even more preferably exceeds 97%.

The mirror material preferably is inert, which ensures that the material does not corrode or otherwise chemically react with transcutaneous gases, sample gas, water or water vapor, gases or liquids related to cleaning, particularly alcohols or alcoholic vapors, or with ambient gases, particularly with oxygen.

The mirror material preferably is thermally well conducting, having a thermal conductivity preferably higher than 30 W/m/K, more preferably exceeding 100 W/m/K, and most preferably exceeding 200 W/m/K, which ensures a uniform mirror temperature. Ideally, the part of the mirror's inner surface forming a part of the measurement chamber is at the same temperature as the remaining measurement chamber surfaces. Hence, there are no cold spots on the mirror and therewith water condensation on the mirror's inner face is prevented.

The mirror may comprise a support material and one or several coatings. For example, the support may consist of a material exhibiting high reflectivity but limited corrosion resistance, where that support is coated with one or several protective coating layers that do not excessively absorb measurement radiation. As another example, the support may consist of a well-deformable material having low reflectivity, which is coated with a layer of inert material having high reflectivity or which is coated with one or more interference layers reflecting measurement radiation.

The mirror preferably consists of a single material. If the mirror consists of a single material, there is no risk of delamination of parts of a coating during deformation of the mirror and of contamination of the measurement chamber.

The mirror material comprises in particular at least one of Au, Ag, Cu, Mo, W, or Al. Preferably, the material consists of one of these elements in high purity form. The material may also be a high-purity alloy predominantly consisting of one of these elements, for example an Au—Cu, Au—Ag, Au—Pd, Cu—Ag—Pd, or Au—Cu—Pt—Ag alloy. In particular, high-purity Au (>99% pure) is a suitable material.

Alternatively, the mirror may comprise or consist of a thermoplastic polymer, such as ABS or POM, with a coating, for example Au or an interference layer. The polymer may be deformed by application of heat, for example by using a heated tool.

In a preferred embodiment of this aspect, the mirror is attached by deformation of the mirror. In this way, the mirror can be attached quickly and in a single step. There is no need of further assembly steps or materials such as adhesives. In particular, the mirror is attached by plastic deformation, preferably by crimping, such that material from a lateral volume of the mirror is displaced into one or several suitable recesses, preferably into an undercut. Preferably, the mirror is attached to an optical module support. The deforming process may result in a tight contact between the mirror and a holder, typically between mirror and optical module support, which allows efficient exchange of thermal energy and thus ensures that all surfaces of the measurement chamber are at homogeneous temperature.

The deformation may be achieved by applying pressure using a suitably formed stamp die, without or with applying heat to support mirror deformation.

After attachment by deformation the boundary between mirror and optical module support may optionally be sealed. A liquid-tight seal may be achieved, for example, by application of a high-viscosity adhesive.

According to a sixth aspect of the invention a sensor for detection of gas, preferably of transcutaneous gas and in particular of gaseous CO₂, is provided. Preferably, the sensor is a sensor as described above.

The sensor comprises at least one radiation source for emitting radiation, at least one detector for detection of radiation emitted by the source, and at least one measurement chamber for receiving the sample gas, preferably arranged on or in an optical module support to form an optical module.

The sensor preferably further comprises a mirror defining a part of the measurement chamber boundaries, preferably arranged on or in an optical module support.

The radiation source, the detector, and the measurement chamber are arranged such that at least a part of the radiation propagates along a path. The path passes through the measurement chamber.

The sensor has a contact face which is directable towards a measuring site and which is intended to be in close contact with the skin of a patient during a transcutaneous measurement.

The sensor comprises at least one gas-access channel enabling sample gas, preferably transcutaneous gas, to migrate from the contact face into the measurement chamber. Preferably, the sensor comprises more than one gas-access channel, for example two, three, or four gas-access channels.

Each gas-access channel has an access opening, which is an opening at one end of a gas-access channel, specifically the end directed towards the contact face. The other opening of a gas-access channel ends in the measurement chamber. An access opening may have the same cross-section as the respective gas-access channel near the access opening. Alternatively, the access-opening may also widen or narrow down towards its contact-face end.

The access opening is located near the contact face of the sensor. The access opening is covered by an access-opening seal, which is permeable for sample gas while being liquid tight or liquid repellent.

The surface of the access-opening seal preferably forms a smooth plane with a surface of a casing, such that the sensor has a smooth contact face. Alternatively, the surface of the access-opening seal slightly stands out from the contact face by not more than a few millimeters, preferably by 0.3 mm or less and more preferably by 0.1 mm or less. Alternatively, the surface of the access-opening seal lies slightly below the plane formed by the contact face, preferably by 0.3 mm or less and more preferably by 0.1 mm or less.

At least a part of the gas-access channel may be arranged within the casing of the sensor and may be formed such that the access opening is placed within or close to the casing wall.

When the surface of the access-opening seal forms a smooth plane with the contact face or only slightly stands above or below the contact face, accumulation of contamination such as contact gel, sweat, particulate matter, or other unwanted substances is largely prevented, while cleaning is easy.

Alternatively, several access openings may be covered by one access-opening seal. For example, all access openings may be covered by one single access-opening seal.

In a preferred embodiment of this aspect, the access-opening seal may comprise a substrate, such as a plate or disk or foil or grid or mesh, having holes or having a porous structure, which optionally is coated or infiltrated with a gas-permeable material.

The access-opening seal may have a specifically designed surface, for example a micro-structured surface or a plasma-treated surface, which renders the surface liquid repellent or liquid tight. This is particularly useful when the access-opening seal does not comprise a coating.

Preferably, the access-opening seal comprises a coating that contains no holes or other openings—Hence it may span across holes or voids in the substrate. Such a seal does not allow liquids or other contamination to enter gas-access channels under normal use conditions. Thus such an access-opening seal in practice is liquid tight. Preferably, the coating is made of a material having a high permeability for sample gas, such that the response time of the sensor remains short, for example of a fluoropolymer such as PTFE, FEP, PFA, or of a further fluoropolymer, or of a polyolefin such as PP, PE, PMP, or PB, or of a silicone such as PDMS, or of a further polymer that preferably has thermoplastic properties and low crystallinity.

Such a coating may reside on one face of the substrate. Alternatively, the coating may additionally extend fully or partly into voids and openings in the substrate. For example, the coating may extend into the upper parts of holes or voids in the substrate, which may anchor the layer in the substrate, or the coating may cover one or both faces of the substrate and extend partly or completely into voids and holes in the substrate.

Alternatively, the coating may exist only in holes or voids of the substrate, with no coating covering a substrate face. In this case, the coating is not necessarily a continuous layer, but preferably, the coating fills all voids in the substrate to an extent that there is no path through the access-opening seal leading neither through substrate material nor through coating material, such that liquids or particulate matter cannot penetrate the seal.

When the access-opening seal comprises a non-continuous coating having pores in locations where the substrate also has holes or pores, it may be made of a material having any or even zero permeability for sample gas. Such an access-opening seal may be liquid repellent but not liquid tight, since liquids may pass through the seal especially when sufficient pressure is applied and when relatively large holes are present in the coating. However, during normal clinical use, this may sufficiently protect the gas-access channel from ingress of liquids or particles, especially when at least during use an additional protecting membrane is present between the access-opening seal and the measurement site on the skin of a patient. Preferably, such a non-continuous coating is hydrophobic and/or oleophobic.

The substrate of the access-opening seal preferably comprises a metallic, ceramic, or polymeric material or consists thereof.

Preferably, the substrate is made of or coated with a material exhibiting good biocompatibility, for example a stainless steel, a titanium alloy, an aluminum oxide, a zirconium oxide, a glass such as a borosilicate glass or a soda-lime glass, or a polymer such as POM, ABS, epoxy, PTFE, FEP, PFA, a further fluoropolymer, PP, PE, PMP, or PB, or a silicone such as PDMS.

An access-opening seal, especially if not liquid tight but only liquid-repellent, may be used in combination with an additional membrane, typically an exchangeable and non-porous membrane spanned over the contact face.

In a further preferred embodiment of this aspect, the substrate of the access-opening seal comprises protrusions and the coating is arranged at least in between the protrusions. The material of the protrusions is more resistive to abrasion than the material of the coating. Alternatively, instead of a substrate having protrusions, the substrate may have depressions. Preferably, the coating is also covering the protrusions, such that the coating forms a smooth surface, in particular if the coating possesses non-stick properties.

Between applications on patients, a sensor needs to be cleaned. Due to the inevitable abrasive effect of repeated cleaning or wiping processes, the coating of the access-opening seal may be affected and damaged with time. Since the protrusions are more resistant to abrasion than the coating, they protect the coating in between the protrusions. When holes in the substrate are located in between the protrusions, those sections of the coating that seal the holes will remain intact and therefore the sealing function is not impaired, even when the upper part of the coating has been worn off.

The protrusions may be formed by hillocks on the substrate of the access-opening seal and may be an intrinsic part of the substrate. The hillocks for example may protrude by 0.1 μm to 500 μm, preferably by few microns to few tens of microns, may have a lateral extension, e.g. a diameter of 1 μm to 500 μm, preferably of few microns to few tens of microns, and may be arranged at an interdistance of 1 μm to 500 μm, preferably of few tens of microns to about 100 microns.

Alternatively, the protrusions may be formed by a rough surface having a surface roughness of Ra>1 μm; in this case the size and arrangement of the protrusions is irregular.

Alternatively, the protrusions may be formed with an irregular shape, for example by the material remaining when a number of depressions are formed into the material. For example, the depressions may be circular in size and created by etching, while correspondingly, the protrusions exist where no depressions were formed.

Preferably, the protrusions are formed such that an undercut is created, which helps anchoring a coating in a substrate. This makes the access-opening seal more durable.

In a further preferred embodiment of this aspect, the access-opening seal may contain at least one anchor, such as one or several “L”- or “T”-shaped legs, allowing mechanical anchoring of the seal. For example, the anchor(s) may be embedded in a potting material, preferably in epoxy. This is particularly useful if the seal contains a non-stick coating, which makes reliable attachment by gluing challenging.

In a further preferred embodiment of this aspect, an additional, liquid-tight membrane may be placed between the access-opening seal and the gas-access channel's access opening. This approach is particularly beneficial when the access-opening seal is porous or contains holes, because the membrane ensures full liquid tightness while a liquid-repellent surface may prevent clogging of the holes or pores in the seal's substrate. In addition, the seal's substrate protects the underlying membrane from mechanical damage.

In a further preferred embodiment of this aspect, the cross-sectional area of a gas-access channel's access opening is greater than the cross-sectional area of a part of the gas-access channel adjacent to its access opening. Preferably, the cross-sectional area of the gas-access channel is rather small, since gas-access channels contribute to the overall gas-accessible volume and since large gas-accessible volumes cause long sensor response times. The access opening preferably may be rather wide (in particular wider than the cross-sectional area of the gas-access channel), such that sample gas is allowed to diffuse from a comparatively wide area into a gas-access channel, which helps to reduce the response time. The area of an access-opening seal is preferably similar in size or wider than the access opening at its interface to the seal.

Preferably, the maximum cross-sectional area of the access opening is at least two times greater than the cross-sectional area of the gas-access channel near its access opening and more preferably at least five to fifty times greater. The access-opening seal's cross-sectional area preferably is only slightly greater than the cross-sectional area of the access opening at its interface to the seal, preferably less than two times greater.

In a further preferred embodiment, the access opening may comprise a star-, or snowflake-, or grid-like structure comprising shallow cavities leading away from the gas-access channel. These cavities help guiding sample gas into the gas-access channel once it has diffused through the access-opening seal. This is particularly advantageous when the access-opening seal is in tight contact with the component(s) comprising the cavities and the gas-access channels, for example, if the access-opening seal is in tight contact with an optical module support. A tight or narrow contact prevents or slows down gas diffusion. Therefore, channel-like cavities provide space for the diffusing gas, which shortens sensor response time. The cavities may also have different shapes/structures; as long as they are connected to the gas-access channel, they help guiding sample gas into the latter.

Preferably, the depth of the described cavities is less than 500 μm, more preferably less than 100 μm, and most preferably less than 50 μm.

In a further preferred embodiment, additionally or alternatively, wide or grid-like shallow cavities may be arranged in the access-opening seal, for example star-, snowflake-, or grid-like cavities as described above for the access opening. This may be easier to produce than if these structures are worked into an optical module or into a corresponding component.

For example, the access-opening seal may contain a cavity slightly less wide than the seal itself, such that after assembling a small gap between the seal and the surroundings of the access opening exists, which promotes gas diffusion into the gas-access channel.

According to a seventh aspect of the invention a sensor for detection of gas, preferably of transcutaneous gas and in particular of gaseous CO₂, is provided. Preferably, the sensor is a sensor as described above.

The sensor comprises an optical module support. The optical module support forms at least a part of the measurement chamber. Further surfaces may define the remaining measurement chamber boundaries. Typically, the main measurement chamber boundaries of the sensor are formed by surface sections of the optical module support, of a mirror, and of radiation entrance and exit windows.

The optical module support has at least one opening, in particular an opening allowing the deposition of a coating at least onto some of the surfaces that serve as measurement chamber surfaces, preferably at least onto all surfaces of the optical module support serving as measurement chamber surfaces. The opening typically is sufficiently wide to allow reliable application of a coating. For example, the opening's area may be similar to or wider than the maximum cross-section of the measurement chamber, which reduces the risk of shadowing during the deposition process.

The sensor preferably comprises a closure component, by means of which said opening in the optical module support can be closed at least partially after application of the coating. Favorably, a mirror as described above may be used as a closure component. Preferably, the closure component exhibits high reflectivity for measurement radiation, preferably exceeding 90%. For example, the mirror may be attached such that a section of its inner face forms a part of the measurement chamber surfaces. Favorably, a small open space between closure component and optical module support may remain open for the purpose of gas migration into and out of the measurement chamber. Preferably, such small open spaces form parts of gas-access channels.

The sensor preferably comprises at least one radiation entrance window. The radiation entrance window preferably is located within the optical module support. The radiation source may be mounted close to the radiation entrance window, for example in a partial or complete source compartment arranged in the optical module support, wherein the detector compartment is separated from the measurement chamber by the radiation entrance window.

The sensor preferably comprises at least one radiation exit window. The radiation exit window preferably is located within the optical module support. The detector for detection of measurement radiation may be mounted close to the radiation exit window, for example in a partial or complete detector compartment being arranged the optical module support, which is separated from the measurement chamber by the radiation exit window.

The optical module support may comprise or consist of a material having a high thermal conductivity, preferably higher than 30 W/m/K, more preferably exceeding 100 W/m/K, and most preferably exceeding 200 W/m/K. Therewith, temperature stabilization of the optical module is easier and temperature within the optical module is more homogeneous.

In a further preferred embodiment of this aspect, if a coating is applied that is reflective for measurement radiation, the optical module support may be manufactured from a material having lower reflectivity. This widens the material choice and allows the optical module support to be manufactured from a material particularly suited for the applied manufacturing process or from a particularly inexpensive material.

At least some of the surfaces of the optical module support also serving as measurement chamber surfaces may receive a reflective coating, with or without additional buffer or barrier layers. Preferably, the reflective coating comprises Ag, Au, Cu, Al, W, or Mo, preferably of a purity exceeding 99%. Preferred deposition methods include electroplating, PVD (sputtering, evaporation), CVD, and ALD.

At least one protective layer may be deposited onto the reflective coating, in particular in the case of coatings which are not sufficiently inert. The protective layer may for example comprise a silicon oxide, a silicon nitride, or an aluminum oxide, which are sufficiently transparent for measurement radiation.

In a further preferred embodiment of this aspect, the optical module support comprises at least a part of the at least one gas-access channel, enabling sample gas, preferably transcutaneous gas, to migrate through the gas-access channel into or out of the measurement chamber.

The sample gas typically is present at the sensor's contact face, for example after diffusion from the skin, and migrates into the gas-access channel(s), preferably via one or several gas-permeable seals and/or membranes which protect each gas-access channel from contamination and clogging, for example via access-opening seals as described above.

Favorably, the optical module support comprises a part of at least one gas-access channel. Preferably, it comprises the main parts of all of the sensor's gas-access channels. Preferably, the channel part does not end at but only close to the contact face, enabling the assembly of an access-opening seal onto the channel's access opening in a way that the seal's surface is at the same level as other parts of the contact face, thus allowing the creation of a smooth contact face without depressions or protrusions.

In this way, one or several gas-access paths leading from near the contact face to the measurement chamber are formed. Molecules of sample gas can so diffuse along these gas-access paths either from the sensor's environment into the measurement chamber or vice versa.

The optical module support may comprise at least one mounting place for a heat source and/or a temperature sensor, which facilitates temperature stabilization of the optical module.

Thus, the optical module may comprise the main elements of the sensor required for gas measurement, such as an optical module support, a radiation source, a detector, a measurement chamber, and gas-access channels. This module may be arranged in a casing, which provides a protective housing and an interface towards the patient and may be connected to driving and read-out electronics.

The optical module support may contain an opening that is closed with a closure component such as a mirror. It further comprises an attachment and sealing zone near that opening.

The attachment and sealing zone is arranged in an area where no gas-access channels are present, typically between the edge of the closure component and openings such as gas-access channels or measurement chamber opening. For example, a mirror can be used as the closure component and its edge is intended to be arranged in an undercut in the optical module support. In this case the attachment and sealing zone is arranged between undercut and gas-access channel openings.

Such an attachment and sealing zone facilitates liquid-tight or gas-tight attachment of the mirror to the optical module support.

In addition, the attachment and sealing zone enables mechanical attachment of a closure component to the optical module support in a way such that a good thermal contact is established between closure component and optical module support. Preferably, a mirror as described above is attached to an optical module support by plastic deformation into an undercut, for example by crimping, such that mirror material is pressed into the undercut and so guarantees a good mechanical contact between mirror and optical module support, which in turn ensures a good thermal contact.

In a further preferred embodiment of this aspect, the optical module support comprises an undercut, preferably near or in an attachment and sealing zone as described above, preferably an undercut leading all around the opening in the optical module, for example in a circle.

A closure component, for example a mirror as described above, can then be attached by plastic deformation. The closure component may seal the opening in the optical module support such that at least no high-viscosity liquids, such as adhesives, and preferably no liquids at all can flow into gas-access channels or the opening.

Alternatively, the closure component may be attached by glueing, preferably using a high-viscosity adhesive.

In a further preferred embodiment of this aspect, the optical module support is formed from a metallic material such as aluminum, brass, bronze, copper, titanium, stainless steel, silver, gold, or from an alloy predominantly containing such metals, or from a ceramic material such as aluminum oxide, zirconium oxide, or aluminum nitride, or from a polymeric material such as epoxy, PEEK, LCP, POM, or ABS.

The optical module support can be manufactured by mechanical machining processes such as drilling, milling, stamping, deep drawing, eroding, grinding or polishing, by optical processes involving lasers such as laser ablation or laser melting, by chemical processes such as etching, by casting or molding, by additive manufacturing processes such as laser melting or laser sintering or thermal sintering, stereolithography, or 3D-printing processes involving two-photon absorption, or by a combination of such processes.

In a further preferred embodiment of this aspect, the sensor has a contact face and the optical module support is arranged within a casing, such that no part of the optical module support forms a part of the contact face. Preferably, at least an access-opening seal as described above is placed between the sensor's contact face and the optical module support.

Preferably, the optical module support is thermally decoupled from the casing. An insulating layer may be arranged between major parts of the optical module support and the casing. The layer may comprise or consist of a material having a low thermal conductivity, preferably less than 10 W/m/K and more preferably less than 1 W/m/K.

In a preferred embodiment of at least one of the sensors as described above, the sensor comprises a measurement chamber, wherein the measurement chamber has surfaces with high reflectivity for measurement radiation. Preferably, all surfaces of the measurement chamber except surfaces of radiation entrance or exit windows are highly reflective, for example, the relevant surfaces of an optical module support and of a mirror.

Preferably, the reflectivity of the measurement chamber surfaces for wavelengths in the range of the wavelengths of measurement radiation exceeds 80%, more preferably exceeds 92%, and even more preferably exceeds 97%.

Preferably, at least some of the surfaces of the measurement chamber are coated with a reflective coating. This allows using materials having insufficient reflectivity to create components such as optical module support or mirror. One or further protective coating(s) may be applied. In this way, materials can be chosen that are particularly suitable for manufacturing or that are inexpensive.

In a preferred embodiment of at least one of the sensors as described above, any space within the sensor, which is not needed for measurement, such as a volume between the optical module and the casing of the sensor, may be filled with a material having no or only little solubility for gas. This may for example be achieved by potting with epoxy. When the empty space is filled with a material having no or only little solubility for gas, exchange of sample gas is reduced even if a leak is present.

The filling process at the same time may be used to create a thermally insulating layer between the optical module support and the casing, as described above.

In a preferred embodiment of at least one of the sensors as described above, the radiation source emits radiation having at least two wavelengths, preferably in the rage of 3.5 to 4.5 μm. Preferably the sensor comprises a wavelength-sensitive element for separating the at least two wavelengths. For example, the wavelength-sensitive element may be an interference filter or an absorption filter.

Preferably, the wavelength-sensitive element is placed close to a detection surface of a detector, for example in between detection surface and radiation exit window. Alternatively, the wavelength-sensitive element may be placed close to the radiation source, for example between source and radiation entrance window. Alternatively, the wavelength-sensitive element may be formed by the radiation entrance or exit window or by a combination of both, for example by coating of suitable faces of the windows.

It may be advantageous to integrate the wavelength-sensing element into a detector to form one component.

In a preferred embodiment of at least one of the sensors as described above, the radiation source is arranged such that at least a part of the emitted radiation propagates through the measurement chamber along a multitude of paths, at least along a first and a second path. At least one of the paths involves a reflection at the mirror. The detector is arranged such that radiation propagating along a first path impinges on a first detection surface and such that radiation propagating along a second path impinges on a second detection surface. A wavelength-sensitive element additionally placed in the optical paths may be helpful in separating the wavelengths of the measurement radiation.

Advantageously, at least a part of the measurement radiation propagates from the source to a detection surface in a non-imaging way.

In a preferred embodiment of at least one of the sensors as described above, the sensor comprises an additional liquid-tight membrane covering areas of the sensor which may not be exposed to liquids. Preferably, the additional membrane covers at least a part of the contact face of the sensor. The additional membrane may be attached to a casing of the sensor.

The additional membrane may be exchangeable.

In a preferred embodiment of at least one of the sensors as described above, the sensor comprises a receiving interface for an additional membrane. The receiving interface favorably is located in the casing and formed such that at least during use of the sensor an additional membrane can be placed over at least a part of the contact face. For example, the additional membrane can be integrated into a patient applicator, in which case the additional membrane could be a disposable membrane preventing a direct contact of skin with the casing. Alternatively, the additional membrane can be a disposable membrane that may remain attached to the casing for one or several consecutive measurements.

In a preferred embodiment of at least one of the sensors as described above, the sensor has a size and shape fitting into a small volume. The sensor (without communication and power supply cables and without connector) preferably fits into a virtual cylinder having a diameter of 30 mm and a height of 20 mm or into a virtual volume of 15 cm³. More preferably, the sensor fits into a cylinder having a diameter of 20 mm and a height of 16 mm or into a virtual volume of 5 cm³. Most preferably, the sensor fits into a cylinder having a diameter of 17 mm and a height of 13 mm or into a virtual volume of 3 cm³.

Furthermore, all measurement radiation rays propagating along optical paths from the radiation source to the detector are confined to this volume. In particular, no optical fibers guiding measurement radiation into or out from the sensor are connected to the sensor.

Preferably, the total length of the shortest complete optical path from the radiation source via the measurement chamber to a detection surface, i.e. of the shortest path followed by measurement radiation, does not exceed 20 mm, preferably does not exceed 10 mm and most preferably does not exceed 5 mm. Such a miniaturized design is advantageous for the manufacture of transcutaneous sensors.

The shortest complete optical path followed by measurement radiation leads not only through gas-accessible volumes like the measurement chamber or unfilled space for example between a radiation entrance window and a source, but also through filled (gas-inaccessible) volumes like radiation entrance or exit windows. Preferably, the length of the section of this shortest complete optical path leading through the measurement chamber relative to the total length of all sections of that path leading through gas-accessible volumes is at least 50%, more preferably at least 60%, and most preferably at least 75%. Such a design improves the reliability of the measurement result.

Furthermore, the sensor preferably generates measurement radiation only within the sensor itself, most preferably within the optical module. Hence, measurement radiation is not transported to the sensor via means such as a waveguide.

Preferably, after an initial warm-up phase, the average electrical power delivered to the sensor during typical measurement conditions is below 5 W, more preferably below 2 W and most preferably below 1 W.

Preferably, the sensor's radiation source is a thermal radiator emitting broad-band black-body-type radiation. Alternatively, the radiation source may also consist of one or several LEDs, for example mid-IR LEDs, or of a tunable or non-tunable laser.

Preferably, the sensor's radiation detector is a detector such as a photoconductor or a photo diode, a (micro-)bolometer, or a pyrometer. The detector is made from a material suitable for the wavelength of the measurement radiation.

According to an eighth aspect of the invention a method for manufacturing of a sensor, in particular of a sensor as described above, is provided.

An optical module support is provided with an opening providing access to at least a part of a measurement chamber. At least a part of the optical module support is then coated through that opening with at least one coating possessing high reflectivity for measurement radiation. The reflective coating may be formed with materials and by methods as described above.

Said opening is then closed with a mirror, such that at least a part of the mirror's surface forms a part of the measurement chamber surfaces.

At least one radiation source for emitting radiation is provided and arranged such that at least a part of that radiation is reflected by the mirror.

Preferably, the mirror is attached by deformation, in particular by plastic deformation. The mirror may for example be deformed by a stamp die. The edge of the mirror may be pressed into an undercut of the optical module support, which fixes it mechanically to the optical module support.

Advantageously, the method comprises the further step of providing a radiation entrance window within the optical module support, which separates the measurement chamber from the sensor's radiation source. The radiation source preferably is mounted in a source compartment arranged within the optical module support. Preferably, the optical module support further comprises a radiation exit window, which separates the measurement chamber from the sensor's detector or detectors. The detector(s) preferably is/are mounted in a detector compartment arranged within the optical module support.

Preferably, at least one radiation source and at least one detector are arranged on or in the optical module support, such that at least a part of the radiation emitted by the source propagates from the source through the measurement chamber and towards a detector, such that it is reflected by the mirror and impinges on a detection surface of a detector.

Advantageously, the method comprises the further step of providing at least a major part of at least one gas-access channel as described above within the optical module support.

The method may comprise the further step of covering a gas-access channel's access opening with an access-opening seal, which is permeable for sample gas, but liquid tight or at least liquid repellent.

The method may comprise the further step of providing a casing as described above. The optical module support may be arranged within the casing.

In a preferred embodiment of the method, the optical module support is formed by a mechanical machining process, by an optical or chemical process, by casting or molding, by additive manufacturing processes, or by a combination of such processes.

The invention is further explained with reference to preferred embodiments and the following drawings which show:

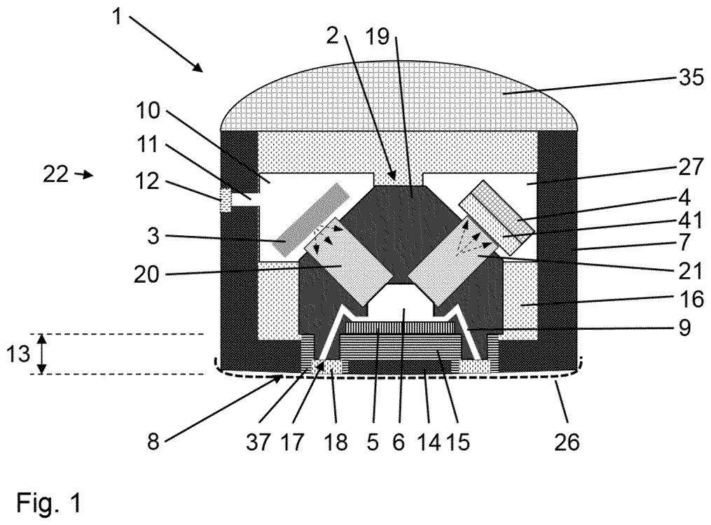

FIG. 1: A schematic cross-sectional view of a first example of a sensor according to the invention;

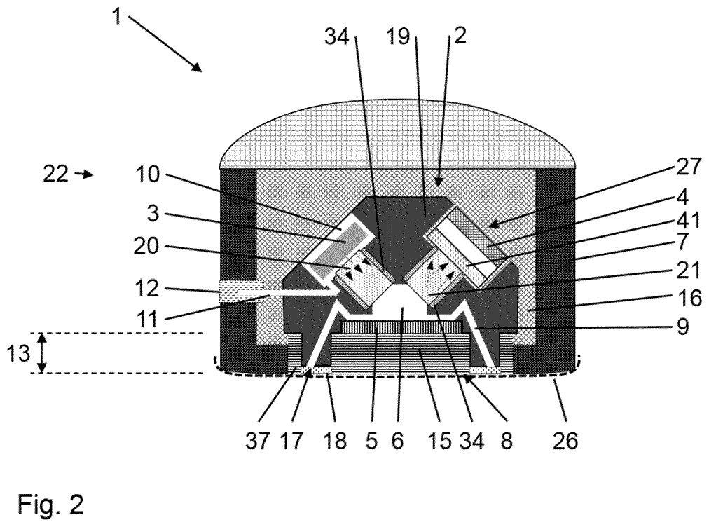

FIG. 2: a schematic cross-sectional view of a further example of a sensor according to the invention;

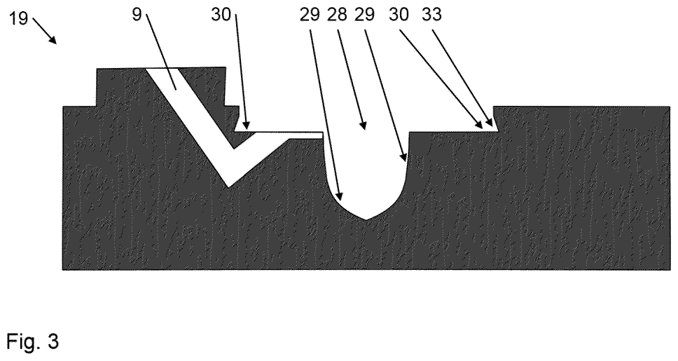

FIG. 3: a schematic cross-sectional view of a part of an optical module support before assembly;

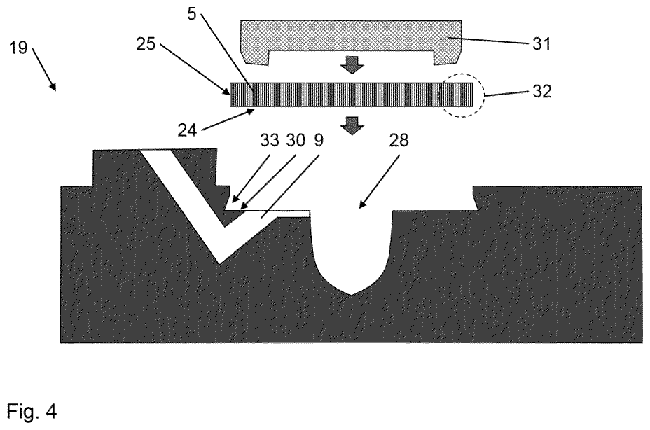

FIG. 4: a schematic cross-sectional view of a part of an optical module support illustrating mirror assembly;

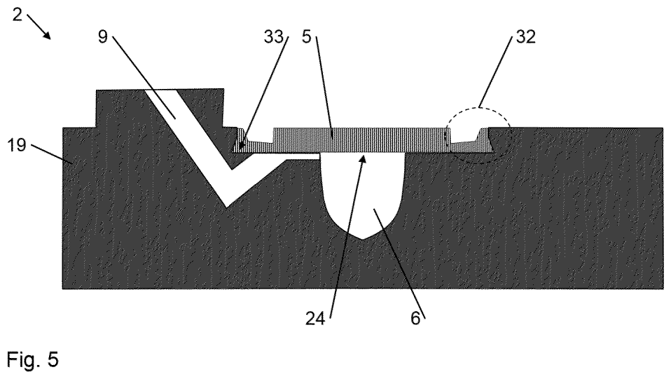

FIG. 5: a schematic cross-sectional view of a part of an optical module after mirror assembly;

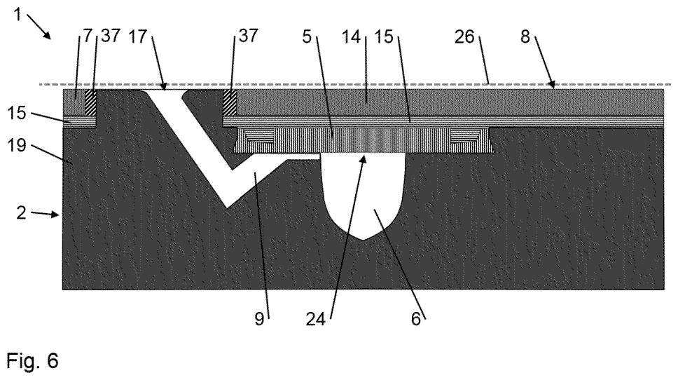

FIG. 6: a schematic cross-sectional view of a part of a further example of a sensor according to the invention;

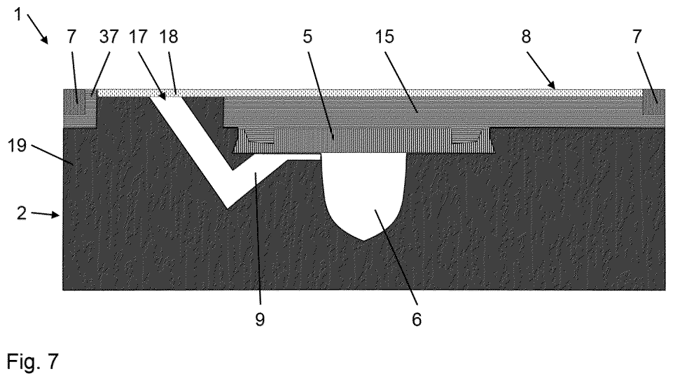

FIG. 7: a schematic cross-sectional view of a part of a further example of a sensor according to the invention;

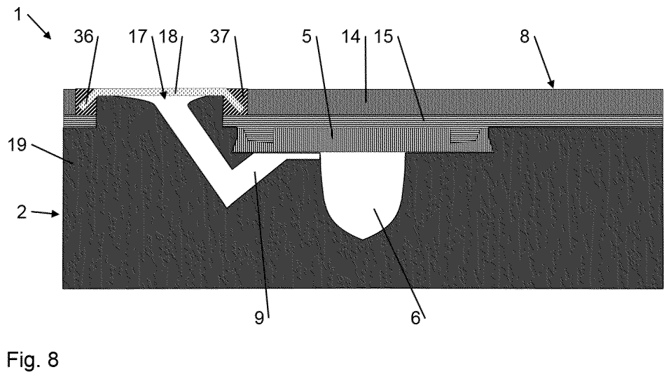

FIG. 8: a schematic cross-sectional view of a part of a further example of a sensor according to the invention;

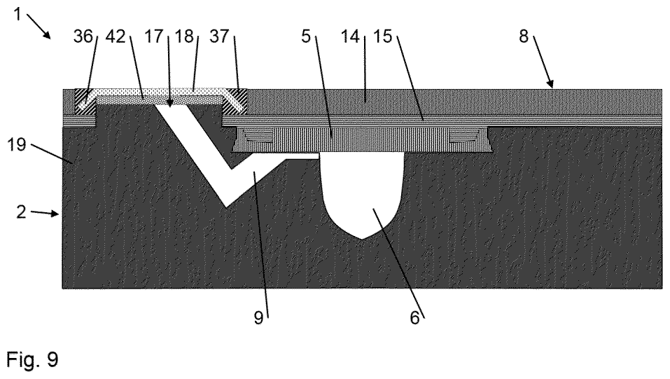

FIG. 9: a schematic cross-sectional view of a part of a further example of a sensor according to the invention;

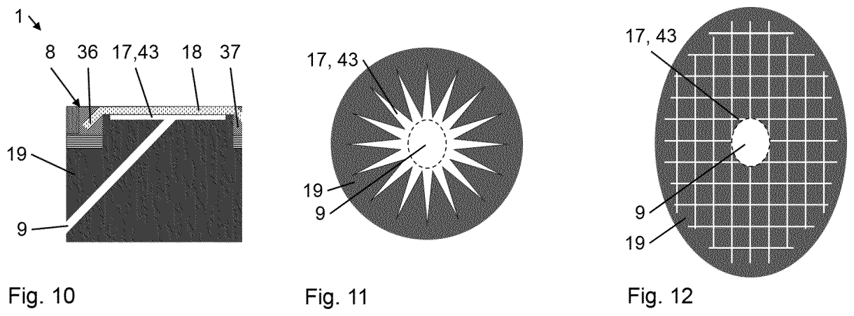

FIG. 10: a schematic cross-sectional view of a part of a further example of a sensor according to the invention;

FIG. 11: a schematic view of a part of an optical module illustrating an example of a gas-access channel's access opening;

FIG. 12: a schematic view of a part of an optical module illustrating a further example of a gas-access channel's access opening;

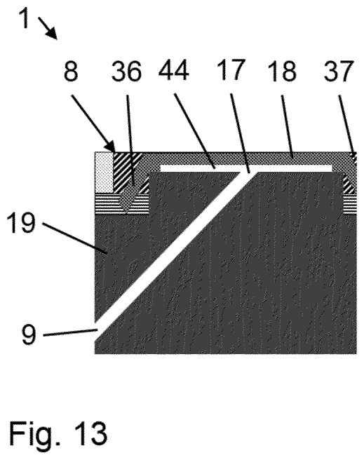

FIG. 13: a schematic cross-sectional view of a part of a further example of a sensor according to the invention;

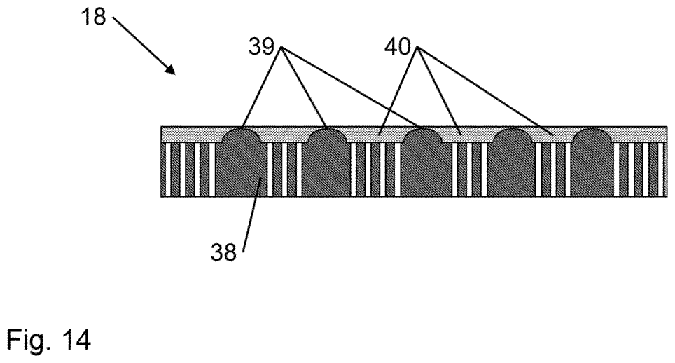

FIG. 14: a schematic cross-sectional view of an example of an access-opening seal;

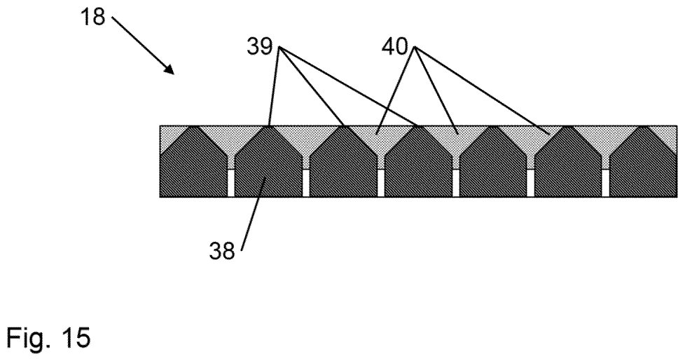

FIG. 15: a schematic cross-sectional view of a further example of an access-opening seal;

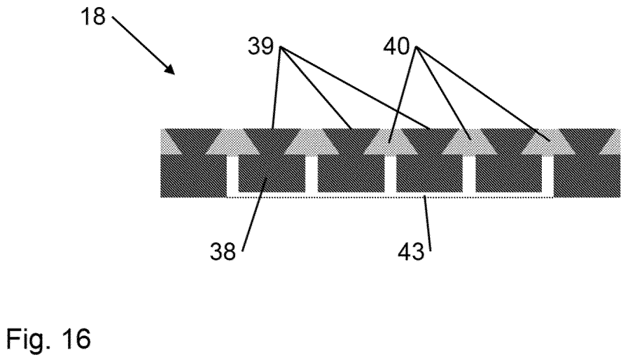

FIG. 16: a schematic cross-sectional view of a further example of an access-opening seal;

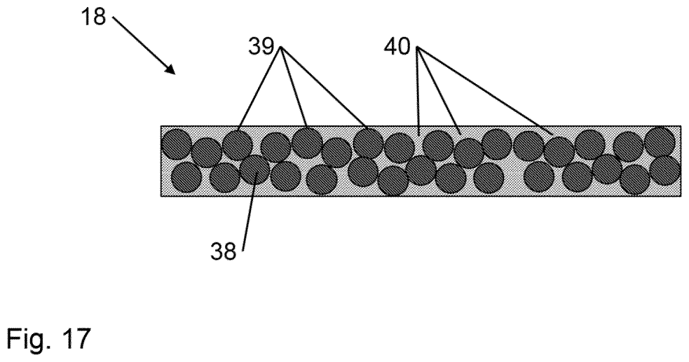

FIG. 17: a schematic cross-sectional view of a further example of an access-opening seal;

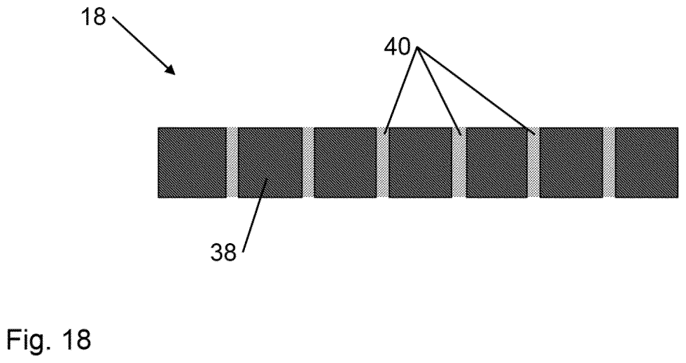

FIG. 18: a schematic cross-sectional view of a further example of an access-opening seal;

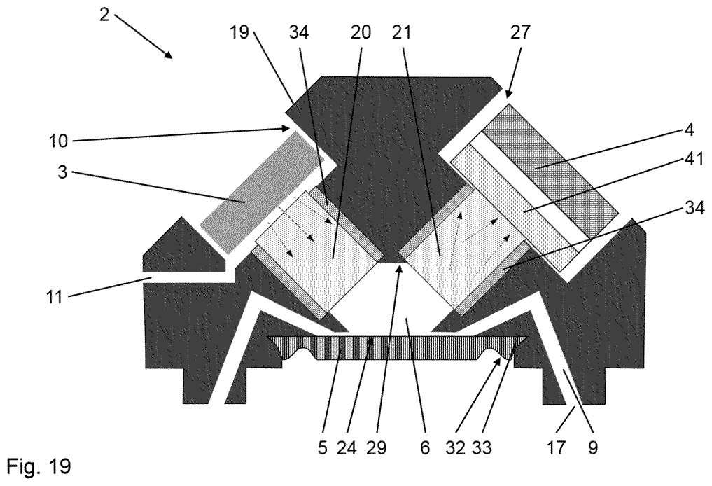

FIG. 19: a schematic cross-sectional view of an example of an optical module.

Identical parts or parts with the same function have the same reference numbers in all figures.

FIG. 1 shows a schematic cross-sectional view of a sensor 1 for detection of gas, in particular a sensor 1 for detection of transcutaneous CO₂. The sensor 1 comprises an optical module 2. The optical module 2 comprises an optical module support 19, a radiation source 3 in the form of a thermal radiator or an LED for emitting radiation, a detector 4 in the form of an infrared photovoltaic or photoconductive detector for detection of radiation emitted by the source 3 and a wavelength-sensitive element 41 formed by an interference filter, a mirror 5 for reflecting the radiation, a measurement chamber 6 for receiving the sample gas, a radiation entrance window 20, and a radiation exit window 21.

The optical module support 19 is for example made of a copper alloy, i.e. of a material having a thermal conductivity of 100 W/m/K or higher. The optical module support 19 does not extend to the contact face 8.

The mirror 5 is for example formed of gold, which is a highly reflecting, deformable, inert, non-porous and/or thermally well conducting material. It is attached to the optical module support 19 by plastic deformation, such that it closes an opening in the optical module support 19 and such that a good thermal contact is established between mirror 5 and optical module support 19.

The radiation source 3, the detector 4, the mirror 5 and the measurement chamber 6 are arranged such that at least a part of the radiation propagates through the measurement chamber 6 along paths involving a reflection at the mirror 5.

The measurement chamber 6 possesses surfaces that are highly reflective for measurement radiation. In this embodiment the high reflectivity is for example achieved by a high-reflectivity coating of gold on these surfaces. The coating may be applied through the opening in the optical module support 19 prior to attachment of the mirror 5.

The sensor 1 further comprises a casing 7, in which the optical module 2 is arranged. The casing 7 is formed of a metallic material (in particular a copper alloy, an aluminum alloy, or a titanium alloy) having a high thermal conductivity of preferably more than 10 W/m/K.

The sensor 1 has a contact face 8 which is directable towards a measuring site and which is intended to be in close contact with the skin of a patient during a transcutaneous measurement.

The sensor 1 has gas-access channels 9 enabling gas to migrate from the contact face 8 into the measurement chamber 6. The gas-access channels 9 are arranged such that they lead neither through nor directly around the mirror 5. The gas-access channels 9 lead from near the contact face 8 into the measurement chamber 6 and are arranged to run along a portion of an inner face 24 (see FIG. 4) of the mirror 5. The portion is chosen such that the gas-access channel 9 does not reach the edge 25 of the mirror 5 (see also FIG. 4). The gas-access channel 9 predominantly is arranged within the optical module support 19.

The radiation source 3 is placed in a source compartment 10. The sensor 1 comprises at least one venting channel 11 leading from the source compartment 10 to the environment 22.

The sensor 1 comprises a liquid tight but gas permeable venting-channel seal 12 for protecting the venting channel 11 from liquids or particles while allowing gas trapped in the source compartment 10 to escape to the environment 22. The seal 12 is for example made of a polymer such as silicone.

The detector 4 is placed in a detector compartment 27.

The mirror 5 is arranged at a distance 13 from the contact face 8 and covered by a part 14 of a casing 7.

The mirror 5 is thermally decoupled from the casing 7. A thermally insulating layer 15 for example made of epoxy is arranged between the mirror 5 and the part 14 of the casing 7.

The gas-access channel 9 ends in an access opening 17 arranged close to the contact face 8 of the sensor 1. The access opening 17 is covered by an access-opening seal 18, which is permeable to gas but liquid tight or liquid repellent. The seal for example is formed by a substrate made of titanium, steel, or glass and a polymeric coating such as a fluoropolymer, a silicone, or a polyolefin. Otherwise empty space between an access-opening seal 18 and casing 7 is filled with a potting material 37, such as epoxy. The access-opening seal 18 and the potting material 37 are formed and arranged such that they form a rather smooth contact face 8 with a part of the casing 7.

Empty space within the sensor's interior, such as between optical module 19 and casing 7, is potted with a potting material 16, in particular with epoxy.

The sensor 1 may comprise an additional membrane 26, at least during use, such that sensor reliability is improved in a wet or in a humid environment. The additional membrane 26 is be attached to a casing 7 of the sensor 1 and exchangeable.

The sensor may furthermore contain a cover or cap 35, preferably made from a polymer, which closes and protects the top part of the sensor.

FIG. 2 shows a schematic cross-section of a further example of a sensor 1. In addition, the optical module 2 comprises radiation-window seals 34 along at least a part of the radiation entrance window 20 and the radiation exit window 21 in order to hinder gas exchange between measurement chamber 6 and source compartment 10 or detector compartment 27.

The sensor 1 further comprises a venting channel 11 leading through the optical module support 19. The venting channel 11 originates in the source compartment. A venting-channel seal 12 allows unwanted gas present in the source compartment 10 to diffuse out of the source compartment 10 and into the sensor's environment 22. The venting channel 11 may extend into the venting-channel seal 12 to some extent.

FIG. 3 shows a schematic cross-sectional view of a part of an optical module support 19 before assembly. The optical module support 19 comprises a major part of a gas-access channel 9 (see FIG. 1). The optical module support 19 comprises an opening 28, which allows the application of a reflective coating 29 onto surfaces defining an important part of the measurement chamber 6 surfaces (see FIG. 1).

The reflective coating 29 is for example made of gold or of a combination of silver with a protective coating. It is highly reflective for measurement radiation.

An attachment and sealing zone 30 is provided on the optical module support 19 on a surface that is to be covered by the mirror 5 (see FIGS. 4 and 5) and that is located between a part of a gas-access channel 9 and an undercut 33 in the optical module support 19.

FIG. 4 shows a schematic cross-sectional view of a part of an optical module support 19 illustrating a method to assemble a mirror 5, where the opening 28 is closed with the mirror 5.

The mirror 5 is placed on the optical module support 19 such that the edge 25 of the mirror 5 will be situated close to the undercut 33 and such that an inner face 24 of the mirror covers a part of the gas-access channel 9. Plastic deformation of the mirror 5 is then achieved by pressing onto the mirror 5 with a stamp die 31, such that material from a lateral volume 32 of the mirror is displaced into the undercut 33 (see FIG. 5).

FIG. 5 shows a schematic cross-sectional view of a part of an optical module 2 after assembly of a mirror 5 onto the optical module support 19 by crimping.

A lateral volume 32 of the mirror 5 is displaced into an undercut 33 in the optical module support 19, such that said mirror 5 is anchored in the undercut 33 and such that a good thermal contact to the optical module support 19 is achieved.

A part of the gas-access channel 9 leads along the mirror's inner face 24 and into the measurement chamber 6.

FIG. 6 shows a schematic cross-sectional view of a part of a further example of a sensor 1.

An optical module 2, comprising at least the optical module support 19 and the mirror 5, is arranged within the casing 7. A thermally insulating layer 15 is provided in between the optical module 2 and casing 7, particularly also between mirror 5 and part 14 of the casing 7.

A gas-access channel 9 runs from the measurement chamber 6 along a part of the inner face 24 of the mirror 5, then through the optical module support 19, not directly around the edge of the mirror 5, to near the contact face 8 of the sensor 1. Gaps between optical module support 19 and casing 7 are filled with potting material 37, which may be different from or the same as the material used as a thermally insulating layer 15. The access opening 17 of the gas-access channel 9 widens near the end of the gas-access channel.

An additional membrane 26 is present near the contact face of the sensor at least during use, which protects the gas-access channel from becoming clogged with contamination such as liquids, pastes, or particles.

FIG. 7 shows a schematic cross-sectional view of a part of a further example of a sensor 1.

In this embodiment, the access opening 17 of the gas access channel 9 is covered by an access-opening seal 18. Any voids between access-opening seal 18 and casing 7 are potted with a potting material 37. Casing 7, potting material 37, and access-opening seal 18 are arranged such that a smooth contact face 8 is formed.

A thermally insulating epoxy layer 15 is arranged between mirror 5 and access-opening seal 18, such that the mirror is thermally decoupled from the contact face 8.

The access-opening seal 18 is much wider than the access opening 17 of the gas-access channel 9. If the sensor possesses one or more further gas-access channels with their individual access openings, the access-opening seal 18 may be used to cover and protect also such further access openings.

FIG. 8 shows a schematic cross-sectional view of a part of a further example of a sensor 1.

The gas-access channel 9 running mostly through the optical module support 19 ends near the contact face 8. The access opening 17 of the gas-access channel 9 widens towards the access-opening seal 18 with which the access opening 17 is covered. The widening of the access opening 17 helps sample gas to migrate into the gas-access channel 9 after having diffused through the access-opening seal 18.

The access-opening seal 18 contains anchors 36. The anchors 36 are embedded in a potting material 37, preferably in epoxy, such that they are mechanically fixed.

FIG. 9 shows a schematic cross-sectional view of a part of a further example of a sensor 1.

The access opening 17 of the gas-access channel 9 is covered with a liquid-tight membrane 42 for example made of a fluoropolymer, a silicone, or a polyolefin, which in turn is covered by an access-opening seal 18 for example made of a metallic, glassy, or ceramic substrate and a polymeric coating.

FIG. 10 shows a schematic cross-sectional view of a part of a further example of a sensor 1.

The access opening 17 is wider than the gas-access channel 9 and comprises a shallow cavity 43 extending along the interface to the access-opening seal 18. This shallow cavity 43 helps sample gas to migrate into the gas-access channel 9 after having diffused through the access-opening seal 18. The shallow cavity 43 or access opening 17 may be formed such that it is present underneath a major part of the access-opening seal 18.

FIG. 11 shows a schematic top view of an example of a part of an optical module support 19, specifically of a gas-access channel's 9 access opening 17. The access opening 17 comprises shallow cavities 43 spreading away from the end of the gas-access channel 9 in a star-like pattern.

Such a star-like shallow cavity 43 is advantageous over a simple shallow cylindrical cavity, because the access-opening seal 18 is mechanically supported over most of its area, while with a cylindrical cavity it is not. This is particularly relevant if the shallow cavity spreads over a large area.

FIG. 12 shows a schematic top view of a further example of a part of an optical module support 19, specifically of a gas-access channel's 9 access opening 17. The access opening 17 comprises shallow cavities 43 arranged in the vicinity of the end of the gas-access channel 9 in a grid-like pattern.

The grid-like arrangement of the shallow cavities 43 supports gas migration towards the gas-access channel 9 also for large areas, i.e., also for large access-opening seals, while the additional gas-accessible volume formed by the shallow cavities 43 remains small. In addition, also large access-opening seals remains mechanically well supported by the optical module support 19.

FIG. 13 shows a schematic cross-sectional view of a part of a further example of a sensor 1.

The access opening 17 of the gas-access channel 9 is not significantly wider than the adjacent part of the gas-access channel 9.

The access-opening seal 18 comprises a shallow cavity 44 extending along a part of the interface to the access-opening seal 18. This shallow cavity 44 helps sample gas to migrate into the gas-access channel 9 after having diffused through the access-opening seal 18 in the same way as the shallow cavities in the access opening 17 as described above (FIGS. 10-12). The shallow cavity 44 may be formed in a major part of the access-opening seal 18 in a star- or grid-like or in any other pattern leading towards the gas-access channel 9.

FIG. 14 shows a schematic cross-sectional view of an example of an access-opening seal 18.

The access-opening seal 18 comprises a substrate 38, which contains holes leading from the upper to the lower face of the substrate. The substrate 38 comprises protrusions 39.

A gas-permeable coating 40 is arranged at least in between the protrusions 39. The material of the protrusions 39 for example is titanium, steel, glass, or a ceramic which is more resistive to abrasion than the material of the permeable coating 40.

Preferably, the gas-permeable coating 40 also covers the protrusions. This is particularly advantageous when the coating 40 for example is a fluoropolymer which possesses non-stick or easy-clean properties.

FIG. 15 shows a schematic cross-sectional view of a further example of an access-opening seal 18.

Here, the gas-permeable coating 40 is arranged at least in between the protrusions 39 and also to some extent or fully within the holes.

FIG. 16 shows a schematic cross-sectional view of a further example of an access-opening seal 18.

The substrate 38 contains larger holes in the upper part of the access-opening seal 18 and smaller holes leading from the bottom of the larger holes to the lower face of the access-opening seal 18. There may be one or several smaller holes in each larger hole. The larger holes may be formed in a conical form such that an undercut is generated. In between the larger holes, the substrate 38 comprises protrusions 39.

A gas-permeable coating 40 is arranged at least in between the protrusions 39 within the larger holes. Hence, the gas-permeable coating 40 in the larger holes is protected from abrasion and at the same time kept in place due to the conical form of the larger holes. The smaller holes may be so small in diameter that the gas-permeable coating 40 does not easily penetrate into them during the coating process. This may enable lower thicknesses of the gas-permeable coating 40, which in turn reduces the diffusion time of sample gas from the measurement site into a gas-access channel.

The access-opening seal 18 additionally comprises shallow cavities 43 in the bottom face, which connect the small holes and lead towards the access opening of a gas-access channel in a fully assembled sensor. The shape of the shallow cavities 43 may be cylindrical, star-like, grid-like, or have any other shape.

FIG. 17 shows a schematic cross-sectional view of a further example of an access-opening seal 18.

The access-opening seal 18 comprises a porous substrate 38, formed by fused particles. There is no direct channel extending from the top to the bottom face of the substrate, or there are only few such channels. Instead, many voids between the constituents of the porous substrate 38 are interconnected and form an arbitrary shaped diffusion channel. The irregular structure of substrate 38 results in protrusions 39.

A gas-permeable coating 40 is arranged at least in between the protrusions 39. The material of the protrusions 39 is more resistive to abrasion than the material of the permeable coating 40. The gas-permeable coating 40 may extend partly or completely into the voids of the substrate.

FIG. 18 shows a schematic cross-sectional view of a further example of an access-opening seal 18.

A gas-permeable coating 40 made of a material as described above is arranged within the holes of the substrate 38, or at least within a part of these holes. The material of the substrate 38 is more resistive to abrasion than the material of the permeable coating 40, hence the substrate protects the coating from damage.

FIG. 19 shows a schematic cross-sectional view of an example of an optical module 2. The optical module 2 comprises an optical module support 19, which preferably is made from a copper alloy which is a thermally well-conducting material. A mirror 5 is attached to the optical module support 19 as described above. Gas-access channels 9 run through the optical module support 19 and between the optical module support 19 and the inner face 24 of the mirror 5. Sample gas present at the access opening 17 of the gas-access channel 9 is able to diffuse into the measurement chamber 6 on a path not directly leading around the edge of the mirror 5.

The optical module 2 further comprises a radiation entrance window 20 and a radiation exit window 21. Radiation-window seals 34 separate measurement chamber 6 from the source compartment 10 and from the detector compartment 27, which both are essentially worked into the optical module support 19, such that gas exchange between measurement chamber 6 and source compartment 10 or detector compartment 27 is strongly limited. Measurement radiation emitted by the radiation source 3 may propagate through the radiation entrance window 20 into the measurement chamber 6, where it will undergo reflection at the mirror 5 and/or the measurement chamber 6 surfaces. Measurement radiation may further propagate through the radiation exit window 21 and through the wavelength-sensitive element 41; thereafter it may be absorbed by a detection surface of a detector 4. The radiation may propagate in a non-imaging way.

The optical module 2 further comprises at least a part of a venting channel 11, through which unwanted gas present in the source chamber 10 may escape.

1.-33. (canceled)

34. A sensor for detection of transcutaneous gas, comprising:
at least one radiation source for emitting radiation;
at least one detector for detection of radiation emitted by the radiation source; and
at least one measurement chamber for receiving the gas to be measured,
the radiation source, the detector, and the measurement chamber being arranged, such that at least a part of the radiation propagates along a path passing through the measurement chamber,
the sensor further comprising a casing, wherein the radiation source, the detector and the measurement chamber are arranged within the casing, the sensor having a contact face which is directable towards a measuring site, wherein the casing comprises a material having a thermal conductivity exceeding 10 W/m/K.

35. The sensor for detection of gas according to claim 34, wherein
the radiation source, the detector, and the measurement chamber are arranged on or in an optical module support.

36. The sensor for detection of gas according to claim 34, wherein
the sensor has at least one gas-access channel enabling the gas to be measured to migrate from the contact face into the measurement chamber.

37. The sensor for detection of gas according to claim 34, wherein the sensor comprises a mirror for reflecting radiation emitted by the radiation source, the mirror being arranged such that at least a part of the radiation propagates along a path involving a reflection at the mirror.

38. A sensor for detection of transcutaneous gas, comprising:
at least one radiation source for emitting radiation;
at least one detector for detection of radiation emitted by the source; and
at least one measurement chamber for receiving the gas to be measured,
the radiation source, the detector, and the measurement chamber being arranged such that at least a part of the radiation propagates along a path passing through the measurement chamber,
the sensor further comprising a casing, wherein the radiation source, the detector and the measurement chamber are arranged within the casing,
the sensor having a contact face which is directable towards a measuring site and
wherein the radiation source is placed in a source compartment and the sensor comprises at least one venting channel leading from the source compartment to the environment.

39. The sensor for detection of gas according to claim 38, wherein the sensor has at least one gas-access channel, enabling the gas to be measured to migrate from the contact face into the measurement chamber.

40. The sensor for detection of gas according to claim 38, wherein the sensor comprises a mirror for reflecting radiation emitted by the radiation source, the mirror being arranged such that at least a part of the radiation propagates along a path involving a reflection at the mirror.

41. The sensor according to claim 38, wherein the sensor comprises a liquid tight venting channel seal for sealing the venting channel.

42. The sensor according to claim 41, wherein the venting channel seal comprises a material selected from at least one of:
a material which is applicable as a liquid or a paste and that cures to a solid, gas-permeable material,
a non-porous but gas-permeable material,
a porous material, and
a supporting material having pores, voids, or holes and a further a gas-permeable layer.

43. A sensor for detection of transcutaneous gas, comprising:
at least one radiation source for emitting radiation;
at least one detector for detection of radiation emitted by the source;
at least one mirror for reflecting the radiation;
at least one measurement chamber for receiving the gas to be measured,
the radiation source, the detector, the mirror, and the measurement chamber being arranged such that at least a part of the radiation propagates along a path involving a reflection at the mirror and passing through the measurement chamber, the sensor having a contact face which is directable towards a measuring site,
wherein the mirror is arranged at a distance from the contact face, and/or the sensor further comprises a casing, wherein the mirror is arranged at a distance from the casing, and the mirror is thermally decoupled from the casing.

44. The sensor according to claim 43, wherein a thermally insulating layer is arranged between the mirror and the casing, the thermally insulating layer comprising a material having a thermal conductivity less than 10 W/m/K.

45. A sensor for detection of transcutaneous gas, comprising
at least one radiation source for emitting radiation;
at least one detector for detection of radiation emitted by the source;
at least one mirror for reflecting the radiation; and
at least one measurement chamber for receiving the gas to be measured,
the radiation source, the detector, the mirror, and the measurement chamber being arranged such that at least a part of the radiation propagates along a path involving a reflection at the mirror and passing through the measurement chamber,
the sensor having a contact face which is directable towards a measuring site and the sensor having at least one gas-access channel enabling the gas to be measured to migrate from the contact face into the measurement chamber,
wherein the walls of the at least one gas-access channel are arranged distant from the mirror's edge and such that they do not lead through the mirror.

46. The sensor according to claim 45, wherein the gas-access channel is arranged to run along a portion of an inner face of the mirror.

47. A sensor for detection of transcutaneous gas, comprising:
at least one radiation source for emitting radiation;
at least one detector for detection of radiation emitted by the source;
at least one mirror for reflecting the radiation; and
at least one measurement chamber for receiving the gas to be measured,
the radiation source, the detector, the mirror and the measurement chamber being arranged such that at least a part of the radiation propagates along a path involving a reflection at the mirror and passing through the measurement chamber, the sensor having a contact face which is directable towards a measuring site wherein the mirror comprises a deformable material.

48. The sensor according to claim 47, wherein the deformable material is at least one of inert, non-porous, thermally well conducting, and highly reflective for measurement radiation.

49. The sensor according to claim 47, wherein the mirror is attached by inelastic deformation of the mirror without adhesives or other fixation members.

50. A sensor for detection of transcutaneous gas, comprising:
at least one radiation source for emitting radiation;
at least one detector for detection of radiation emitted by the source; and
at least one measurement chamber for receiving the gas to be measured,
the radiation source, the detector, and the measurement chamber being arranged such that at least a part of the radiation propagates along a path passing through the measurement chamber,
the sensor having a contact face which is directable towards a measuring site and the sensor having at least one gas-access channel enabling the gas to be measured to migrate from the contact face into the measurement chamber wherein the gas-access channel comprises an access opening,
wherein the access opening is covered by an access-opening seal, which is permeable for the gas to be measured, but liquid tight or liquid repellent,
wherein the access opening is located near the contact face of the sensor such that the surface of the access-opening seal does not stand above or lie below the contact face by more than 0.3 mm.

51. The sensor according to claim 50, wherein the access-opening seal comprises a substrate having holes or being porous and a gas-permeable coating or filling.

52. The sensor according to claim 51, wherein the substrate of the access-opening seal comprises protrusions, and wherein the permeable coating or filling is arranged at least in between the protrusions, wherein the material of the protrusions is more resistive to abrasion than the material of the permeable coating.

53. The sensor according to claim 50, wherein the access-opening seal contains at least one anchor that is mechanically fixed.

54. The sensor according to claim 50, wherein a liquid-tight membrane is placed between the access-opening seal and the access opening of the gas-access channel.

55. The sensor according to claim 50, wherein the cross-sectional area of a gas-access channel has an at least local maximum at said access opening.

56. The sensor according to claim 55, wherein the cross-sectional area of a gas-access channel at a location next to its access opening is smaller than the cross-sectional area of said access opening.

57. The sensor according to claim 50, wherein the access-opening comprises shallow cavities leading away from the gas-access channel.

58. The sensor according to claim 50, wherein the access-opening seal comprises shallow cavities running towards the gas-access channel.

59. A sensor for detection of transcutaneous gas, the sensor comprising:
at least one radiation source for emitting radiation;
at least one detector for detection of radiation emitted by the source; and
at least one measurement chamber for receiving the gas to be measured,
the radiation source, the detector, and the measurement chamber being arranged such that at least a part of the radiation propagates along a path passing through the measurement chamber,
wherein the sensor comprises an optical module support, wherein that optical module support forms a part of the measurement chamber and comprises an opening.

60. The sensor according to claim 59, wherein the opening allows the deposition of a reflective coating onto at least a part of the measurement chamber surfaces.

61. The sensor according to claim 59, wherein the sensor comprises a closure component with which the opening can be closed after deposition of a reflective coating.

62. The sensor according to claim 59, wherein the optical module support comprises a material having a thermal conductivity of at least 30 W/m/K.

63. The sensor according to claim 59, wherein the optical module support comprises at least a part of at least one gas-access channel, enabling the gas to be measured to migrate through the gas-access channel into the measurement chamber.

64. The sensor according to claim 59, wherein the optical module support comprises an attachment and sealing zone near said opening, enabling mechanical attachment of the closure component to the optical module support with good thermal contact.

65. The sensor according to claim 59, wherein the optical module support comprises an undercut, in which a lateral volume of the closure component can be arranged.

66. The sensor according to claim 59, wherein the optical module support is formed from a material selected from the group consisting of brass, bronze, stainless steel, pure aluminum, alloyed aluminum, copper, titanium, silver, gold, aluminum oxide, zirconium oxide, aluminum nitride, epoxy, PEEK, LCP, POM, and ABS.

67. The sensor according to claim 59, wherein the sensor has a contact face and wherein the optical module support is arranged within a casing, such that no part of the optical module support forms a part of the contact face.

68. The sensor according to claim 34, wherein the measurement chamber is confined by surfaces of which at least some have high reflectivity for measurement radiation.

69. A method for manufacturing a sensor, comprising the steps of:
providing an optical module support with an opening revealing at least a part of a measurement chamber;
coating at least a part of the optical module support with at least one reflective coating through that opening;
closing said opening with a mirror after application of the reflective coating, such that a part of the mirror's inner face forms a part of the measurement chamber's surfaces; and
arranging at least one radiation source for emitting radiation such that at least a part of that radiation is reflected by the mirror.

70. The method according to claim 69, wherein for attachment of the mirror is deformed.

71. The method according to claim 70, wherein the mirror is inelastically deformed.

72. The method according to claim 69, comprising the further step of arranging on or in the optical module support at least one detector for detecting radiation emitted by the radiation source, the detector being arranged such that at least a part of the radiation emitted by the radiation source propagates through the measurement chamber and impinges on a detection surface of the detector.

73. The method according to claim 69, comprising the further step of providing at least a major part of at least one gas-access channel within the optical module support, the gas-access channel leading into the measurement chamber.

74. The method according to claim 73, comprising the further step of covering an access opening of the gas-access channel with an access-opening seal, which is permeable for the gas to be measured, but liquid tight or liquid repellent.

75. The method according to claim 69, comprising the further steps of providing a casing with a contact face which is directable towards a measuring site, arranging the optical module support within the casing.

76. The method according to claim 75, comprising the further steps of providing at least one gas-access channel, with an access-opening seal, such that the access-opening seal forms a smooth contact face with the surface of the casing.
